(19) United States
(12) Patent Application Publication
(10) Pub. No.: US 2011/0052543 A1
(43) Pub. Date: Mar. 3, 2011

(54) NOVEL BACTERIOPHAGE AND ANTIBACTERIAL COMPOSITION COMPRISING THE SAME

(75) Inventors: Soo An Shin, Seoul (KR); Min Tae Park, Seoul (KR); Hyang Choi, Anyang-si (KR); Young Wook Cho, Seoul (KR); In Hye Kang, Suwon-si (KR); Su Jin Choi, Daegu (KR)

(73) Assignee: CJ CHEILJEDANG CORPORATION, Seoul (KR)

(21) Appl. No.: 12/875,879

(22) Filed: Sep. 3, 2010

Related U.S. Application Data
(60) Provisional application No. 61/239,743, filed on Sep. 3, 2009.

(51) Int. Cl. A61K 35/76; C12N 7/00; A61P 31/04; A01N 63/00; A01P 1/00

(52) U.S. Cl. 424/93.6; 435/235.1

(57) ABSTRACT

Disclosed herein are is a novel bacteriophage which has specific bactericidal activity against one or more Salmonella bacteria selected from the group consisting of Salmonella Enteritidis, Salmonella Typhimurium, Salmonella Gallinarum, and Salmonella Pullorum without affecting beneficial bacteria. Disclosed are also compositions, animal feeds or drinking water, cleaners and sanitizers for preventing and treating the infectious diseases caused by Salmonella Enteritidis, Salmonella Typhimurium, Salmonella Gallinarum or Salmonella Pullorum including salmonellosis, Salmonella food poisoning, Fowl Typhoid, and Pullorum disease or for controlling the salmonella bacteria.

### TECHNICAL FIELD

The present invention relates to a novel bacteriophage and antibacterial composition comprising the same.

### BACKGROUND ART

Salmonella is a genus of the family Enterobacteriaceae, characterized as Gram-negative, facultatively anaerobic, non spore-forming, rod-shaped bacteria, and most strains are motile by flagella. Salmonella has an average genomic GC content of 50-52%, which is similar to that of Escherichia coli and Shigella. The genus Salmonella is a pathogenic microorganism that causes infections in livestock as well as in humans. Serological division has it that Salmonella enterica, a species of Salmonella bacterium, has a variety of serovars including Gallinarum, Pullorum, Typhimurium, Enteritidis, Typhi, Choleraesuis, and derby. Of them, Salmonella Gallinarum and Pullorum are fowl-adapted pathogens, Salmonella Typhi is a human-adapted pathogen, Salmonella Choleraesuis and Salmonella derby are swine-adapted pathogens, and Salmonella Enteritis and Salmonella Typhimurium are pathogenic for humans and animals. Each serovar causes illness in the respective species, resulting in tremendous damage to farmers or consumers.

A disease of domestic birds caused by Salmonella bacterium is Fowl Typhoid (FT), which is caused by a pathogen, Salmonella Gallinarum (hereinafter, referred to as “SG”). Fowl Typhoid (FT) is a septicemic disease of domestic birds such as chicken and turkey, and the course may be acute or chronic with high mortality. A recent report has had it that Fowl Typhoid frequently occurs in Europe, South America, Africa, and Southeast Asia, with damages increasing every year. Outbreaks of FT in South Korea have been reported since 1992 and economic losses caused by FT in brown, egg-laying chickens are very serious (Kwon Yong-Kook. 2000 annual report on avian diseases. Information publication by National Veterinary Research & Quarantine Service. March, 2001; Kim Ae-Ran et al., The prevalence of pullorum disease-fowl typhoid in grandparent stock and parent stock in Korea, 2003, Korean J Vet Res(2006) 46(4): 347-353).

Pullorum disease is also caused by a strain of the Salmonella bacteria, Salmonella Pullorum (hereinafter, referred to as “SP”). Pullorum disease occurs in any age or season, but young chickens are particularly susceptible to the disease. During the past century, it has been a serious disease among young chickens at 1-2 weeks of age or younger. Since the 1980s, the occurrence has greatly decreased. However, it has been growing since the mid-1990s (Kwon Yong-Kook. 2000 annual report on avian diseases. Information publication by National Veterinary Research & Quarantine Service. March, 2001; Kim Ae-Ran et al., The prevalence of pullorum disease-fowl typhoid in grandparent stock and parent stock in Korea, 2003, Korean J Vet Res(2006) 46(4): 347-353).

In South Korea, outbreaks of Fowl Typhoid and Pullorum disease have been increasing since the 1990s, inflicting economic damages on farmers. For this reason, a live attenuated SG vaccine has been used in broilers for the prevention of Fowl Typhoid from 2004 (Kim Ae-Ran et al., The prevalence of pullorum disease-fowl typhoid in grandparent stock and parent stock in Korea, 2003, Korean J Vet Res(2006) 46(4): 347-353). Its efficacy is doubtful, and the live vaccine is not allowed to be used for layers because of the risk of egg-transmitted infections. Unfortunately, there are still no commercially available preventive strategies against Pullorum disease, unlike Fowl Typhoid. Thus, there is an urgent need for new ways to prevent Fowl Typhoid and Pullorum disease.

Meanwhile, Salmonella Enteritidis (hereinafter, referred to as “SE”) and Salmonella Typhimurium (hereinafter, referred to as “ST”) are zoonotic pathogens, which show no host specificity, unlike SG or SP (Zoobises Report; United Kingdom 2003).

SE and ST are causative of salmonellosis in poultry, pigs, and cattle. Salmonellosis, caused by Salmonella bacteria, is an acute or chronic infection of the digestive tract in livestock, and shows the major symptoms of fever, enteritis, and septicemia, occasionally pneumonia, arthritis, abortion, and mastitis. Salmonellosis occurs worldwide, and most frequently during the summer months (T. R. Callaway et al. Gastrointestinal microbial ecology and the safety of the food supply as related to Salmonella. J Anim Sci 2008.86:E163-E172). In cattle, typical symptoms include loss of appetite, fever, dark brown diarrhea or bloody mucous in stool. The acute infection in calves leads to rapid death, and the infection during pregnancy leads to fetal death due to septicemia, resulting in premature abortion (www.livestock.co.kr). In pigs, salmonellosis is characterized clinically by three major syndromes: acute septicemia, acute enteritis, and chronic enteritis. Acute septicemia occurs in 2˜4-month-old piglets, and death usually occurs within 2˜4 days after onset of symptoms. Acute enteritis occurs during the fattening period, and is accompanied by diarrhea, high fever, pneumonia, and nervous signs. Discoloration of the skin may occur in some severe cases. Chronic enteritis is accompanied by continuing diarrhea (www.livestock.co.kr).

Once an outbreak of salmonellosis by SE and ST occurs in poultry, pigs, and cattle, it is difficult to cure only with therapeutic agents. The reasons are that Salmonella bacteria exhibits a strong resistance to various drugs and live in cells that are impermeable to antibiotics upon the occurrence of clinical symptoms. Up to now, there have been no methods for effectively treating salmonellosis caused by SE and ST, including antibiotics (www.lhca.or.kr).

As in livestock, SE and ST cause infections in humans via livestock and their products, leading to salmonella food poisoning. Intake of infected, improperly cooked livestock products (e.g., meat products, poultry products, eggs and by-products) infects humans. Salmonella food poisoning in humans usually involves the prompt onset of headache, fever, abdominal pain, diarrhea, nausea, and vomiting. The symptoms commonly appear within 6-72 hours after the ingestion of the organism, and may persist for as long as 4-7 days or even longer (NSW+HEALTH. 2008.01.14.).

According to a report by the CDC (The Centers for Disease Control and Prevention, USA), 16% of human food poisoning outbreaks between 2005 and 2008 were attributed to Salmonella bacteria, with SE and ST responsible for 20% and 18% thereof, respectively. With respect to salmonella food poisoning in humans between 1973 and 1984, the implicated food vehicles of transmission were reportedly chicken (5%), beef (19%), pork (7%), dairy products (6%), and turkey (9%). In 1974-1984, the bacterial contamination test on broilers during the slaughter process showed 35% or more of salmonella incidence. In 1983, salmonella was isolated in 50.6% of chicken, 68.8% of turkey, 60% of goose, 11.6% of pork, and 1.5% of beef. Further, a survey carried out in 2007 reported that salmonella was found in 5.5% of raw poultry meat and 1.1% of raw pork. In particular, it was revealed that SE commonly originated from contaminated egg or poultry meat, and ST from contaminated pork, poultry meat, and beef (www.cdc.gov) (Centers for Disease Control and Prevention (CDC)). For example, food poisoning caused by SE has rapidly increased in the US, Canada, and Europe since 1988, and epidemiological studies demonstrated that it was attributed to eggs or egg-containing foods (Agre-Food Safety Information Service(AGROS). Domestic and foreign food poisoning occurrence and management trend. 2008. 02). A risk assessment conducted by FAO and WHO in 2002 noted that the human incidence of salmonellosis transmitted through eggs and poultry meat appeared to have a linear relationship to the observed Salmonella prevalence in poultry. This means that, when reducing the prevalence of Salmonella in poultry, the incidence of salmonellosis in humans will fall (Salmonella control at the source; World Health Organization. International Food Safety Authorities Network (INFOSAN) Information Note No. 03/2007). Recently, fears about food safety have been spurred by outbreaks of salmonella from products as varied as peanuts, spinach, tomatoes, pistachios, peppers and, most recently, cookie dough (Jane Black and Ed O'Keefe. Overhaul of Food Safety Rules in the Works. Washington Post Staff Writers Wednesday, Jul. 8, 2009).

For these reasons, Salmonella infections must be reported in Germany (§6 and §7 of the German law on infectious disease prevention, Infektionsschutzgesetz). Between 1990 and 2005 the number of officially recorded cases decreased from approximately 200,000 cases to approximately 50,000. It is estimated that every fifth person in Germany is a carrier of Salmonella. In the USA, there are approximately 40,000 cases of Salmonella infection reported each year (en.wikipedia.org/wiki/Salmonella*cite_note-2).

Therefore, there is an urgent need to control SE and ST, which cause salmonellosis in livestock and humans. The collaborative efforts of USDA and FDA have developed a number of effective strategies to prevent salmonellosis that causes over 1 million cases of food-borne illness in the United States. Among them is a final rule, issued by the FDA, to reduce the contamination in eggs. The FDA will now require that egg producers test regularly for lethal salmonella during egg production, storage and shipment. As a result, an estimated 79,000 illnesses and 30 deaths due to SE contaminated eggs will be avoided each year (Jane Black and Ed O'Keefe. Overhaul of Food Safety Rules in the Works. Washington Post Staff Writers Wednesday, Jul. 8, 2009). In Denmark, conservative estimates from a cost benefit analysis comparing Salmonella control costs in the production sector with the overall public health costs of salmonellosis suggest that Salmonella control measures saved Danish society US$ 14.1 million in the year 2001 (Salmonella control at the source. World Health Organization. International Food Safety Authorities Network(INFOSAN) Information Note No. 03/2007).

Meanwhile, bacteriophage is a specialized type of virus that infects and destroys only bacteria, and can self-replicate only inside host bacteria. Bacteriophage consists of genetic material in the form of single or double stranded DNA or RNA surrounded by a protein shell. Bacteriophages are classified into three basic structural forms: an icosahedral (twenty-sided) head with a tail; an icosahedral head without a tail; and a filamentous form. Based on their tail structure, the most abundant form bacteriophages, which have an icosahedral head with a tail, are further divided into: Myoviridae, Siphoviridae, and Podoviridae, which are characterized by contractile, long non-contractile, and short noncontractile tails, respectively. Bacteriophages having an icosahedral head without a tail are divided based on their head shape and components, and the presence of shell. Filamentous bacteriophages having DNA as their genetic material are divided based on their size, shape, shell, and filament components (H. W. Ackermann. Frequency of morphological phage descriptions in the year 2000; Arch Virol (2001) 146:843-857; Elizabeth Kutter et al. Bacteriophages biology and application; CRC press).

During infection, a bacteriophage attaches to a bacterium and inserts its genetic material into the cell. After this a bacteriophage follows one of two life cycles, lytic or lysogenic. Lytic bacteriophages take over the machinery of the cell to make phage components. They then destroy or lyse the cell, releasing new phage particles. Lysogenic bacteriophages incorporate their nucleic acid into the chromosome of the host cell and replicate with it as a unit without destroying the cell. Under certain conditions, lysogenic phages can be induced to follow a lytic cycle.

After the discovery of bacteriophages, a great deal of faith was initially placed in their use for infectious-disease therapy. However, when broad spectrum antibiotics came into common use, bacteriophages were seen as unnecessary due to a specific target spectrum. Nevertheless, the misuse and overuse of antibiotics resulted in rising concerns about antibiotic resistance and harmful effects of residual antibiotics in foods. In particular, antimicrobial growth promoter (AGP), added to animal feed to enhance growth, is known to induce antibiotic resistance, and therefore, the ban of using antimicrobial growth promoter (AGP) has been recently introduced. In the European Union, the use of all antimicrobial growth promoters (AGPs) was banned from 2006. South Korea has banned the use of some AGPs, and is considering restrictions on the use of all AGPs in future.

These growing concerns about the use of antibiotics have led to a resurgence of interest in bacteriophage as an alternative to antibiotics. Seven bacteriophages for control of E. coli 0157:H are disclosed in U.S. Pat. No. 6,485,902 (Use of bacteriophages for control of Escherichia coli 0157, issued in 2002). Two bacteriophages for control of various microorganisms are disclosed in U.S. Pat. No. 6,942,858 (issued to Nymox in 2005). Many companies have been actively trying to develop various products using bacteriophages. EBI food system (Europe) developed a food additive for preventing food poisoning caused by Listeria monocytogenes, named Listerix-P100, which is the first bacteriophage product approved by the US FDA. A phage-based product, LMP-102 was also developed as a food additive against Listeria monocytogenes, approved as GRAS (Generally Regarded As Safe). In 2007, a phage-based wash produced by OmniLytics was developed to prevent E. coli 0157 contamination of beef during slaughter, approved by USDA's Food Safety and Inspection Service (FSIS). In Europe, Clostridium sporogenes phage NCIMB 30008 and Clostridium tyrobutiricum phage NCIMB 30008 were registered as a feed preservative against Clostridium contamination of feed in 2003 and 2005, respectively. Such studies show that research into bacteriophages for use as antibiotics against zoonotic pathogens in livestock products is presently ongoing.

However, most of the phage biocontrol studies have focused on the control of E. coli, Listeria, and Clostridium. Salmonella is also a zoonotic pathogen, and damages due to this pathogen are not reduced. As mentioned above, since SE and ST exhibit multiple drug resistance, nationwide antimicrobial resistance surveillance has been conducted in South Korea under the Enforcement Decree of the Act on the Prevention of Contagious Disease (Executive Order 16961), Enforcement ordinance of the Act on the Prevention of Contagious Disease (Ministry of Health and Welfare's Order 179), and Organization of the National Institute of Health (Executive Order 17164). Accordingly, there is a need for the development of bacteriophages to control Salmonella. The foregoing discussion is solely to provide background information of the invention and do not constitute an admission of prior art.

### SUMMARY

One aspect of the present invention relates to an isolated bacteriophage, belonging to the family Siphoviridae of morphotype B1, with a specific bactericidal activity against one or more Salmonella bacteria selected from the group consisting of Salmonella Enteritidis, Salmonella Typhimurium, Salmonella Gallinarum, and Salmonella Pullorum, characterized by one of the following properties: 1) the bacteriophage has a total genome size of 41˜43 kbp; 2) the bacteriophage contains as a part of the genome thereof at least one nucleic acid sequence selected from among SEQ ID NO. 1, SEQ ID NO. 2, SEQ ID NO. 3, SEQ ID NO. 4, SEQ ID NO. 5, SEQ ID NO. 6, and SEQ ID NO. 7; and 3) the bacteriophage has structural proteins ranging in size from 37-40 kDa, 62-65 kDa, 51-54 kDa and 11-13 kDa.

According to some embodiments, the bacteriophage may have a morphological structure composed of an isometric capsid and a long non-contractile tail.

According to some embodiments, when PCR is performed in a presence of a primer set selected from among SEQ ID NOS. 8 and 9, SEQ ID NOS. 10 and 11, SEQ ID NOS. 12 and 13, SEQ ID NOS. 14 and 15, SEQ ID NOS. 16 and 17, SEQ ID NOS. 18 and 19, and SEQ ID NOS. 20 and 21, with the genome of the bacteriophage serving as a template, each PCR product is 500 by ˜3 kbp long.

According to some embodiments, the bacteriophage may show at least one of the following properties; 1) tolerance to a range of from pH 3.0 to pH 11.0; 2) tolerance to a heat range of from 37° C. to 70° C.; and 3) tolerance to desiccation under a condition of 60° C./120 min.

According to some embodiments, the bacteriophage may be identified by accession number KCCM11028P.

Another aspect of the present invention relates to a composition for prevention or treatment of infectious diseases caused by one or more Salmonella strains selected from the group consisting of Salmonella Enteritidis, Salmonella Typhimurium, Salmonella Gallinarum, and Salmonella Pullorum, comprising the foregoing bacteriophage as an active ingredient.

According to some embodiments, the infectious diseases may be salmonellosis and salmonella food poisoning when caused by Salmonella enteritidis or Salmonella Typhimurium, Fowl typhoid when caused by Salmonella Gallinarum and pullorum when caused by Salmonella Pullorum.

According to some embodiments, the composition may be used as an antibiotic.

Still another aspect of the present invention relates to an animal feed or drinking water, comprising the foregoing bacteriophage as an active ingredient.

Still another aspect of the present invention relates to a sanitizer and cleaner, comprising the foregoing bacteriophage as an active ingredient.

Still another aspect of the present invention relates to a method for preventing or treating infectious diseases caused by one or more Salmonella strains selected from the group consisting of Salmonella Enteritidis, Salmonella Typhimurium, Salmonella Gallinarum, and Salmonella Pullorum, comprising administering the foregoing bacteriophage to animals in need thereof.

Still another aspect of the present invention relates to a method for preventing or treating infectious diseases caused by one or more Salmonella strains selected from the group consisting of Salmonella Enteritidis, Salmonella Typhimurium, Salmonella Gallinarum, and Salmonella Pullorum, comprising administering the foregoing bacteriophage to animals in need thereof.

### DETAILED DESCRIPTION OF THE EMBODIMENTS

Leading to the present invention, intensive and thorough research into bacteriophages, isolated from natural sources, which infect the poultry pathogen salmonella, conducted by the present inventors, aiming to overcome problems occurring upon the misuse or overuse of broad spectrum antibiotics, such as the advent of drug or multiple drug resistant bacteria, drug residues, etc., resulted in the finding that some isolated bacteriophages have a specific bactericidal activity against Salmonella Enteritidis (SE), Salmonella Typhimurium (ST), Salmonella Gallinarum (SG) and Salmonella Pullorum (SP) with no influences on beneficial bacteria, in addition to showing excellent acid- and heat-resistance and desiccation tolerance, as identified for the morphorlogical, biochemical and genetic properties thereof, and thus that some bacteriophages can be used as active ingredients of compositions for the prevention and treatment of Salmonella Enteritidis- or Salmonella Typhimurium-mediated diseases, such as livestock salmonellosis and Salmonella food poisoning, and Salmonella Gallinarum- or Salmonella Pullorum-mediated diseases, particularly, Fowl Typhoid and Pullorum disease. Also, bacteriophages according to some embodiment of the present invention can be applied to various products for the control of Salmonella bacteria, including livestock feed additives, drinking water for livestock, barn sanitizers, and cleaners for meal products.

It is one aspect of the present invention to provide a bacteriophage which has a specific bactericidal activity against one or more Salmonella bacteria selected from the group consisting of Salmonella Enteritidis, Salmonella Typhimurium, Salmonella Gallinarum, and Salmonella Pullorum.

It is another aspect of the present invention to provide a composition for the prevention or treatment of infectious diseases caused by one or more Salmonella bacteria selected from the group consisting of Salmonella Enteritidis, Salmonella Typhimurium, Salmonella Gallinarum, and Salmonella Pullorum, comprising the bacteriophage as an active ingredient. Preferably, the composition is used as an antibiotic.

It is a further aspect of the present invention to provide a livestock feed and drinking water for livestock,

It is still a further aspect of the present invention to provide a cleaner or a sanitizer, comprising the bacteriophage as an active ingredient.

It is still another aspect of the present invention to provide a method for preventing or treating salmonellosis or salmonella food poisoning caused by Salmonella Enteritidis or Salmonella Typhimurium using the composition comprising the bacteriophage as an active ingredient. Also, the present invention provides a method for preventing or treating fowl typhoid and pullorum disease caused by Salmonella Gallinarum or Salmonella

The novel bacteriophages of according to some embodiments of the present invention has a specific bactericidal activity against one or more Salmonella strain selected from the group consisting of Salmonella Enteritidis, Salmonella Typhimurium, Salmonella Gallinarum, and Salmonella Pullorum, in addition to showing excellent acid- and heat-resistance and desiccation tolerance. Hence, the novel bacteriophage according to some embodiments of the present invention can be used for preventing or treating infectious diseases caused by Salmonella Enteritidis, Salmonella Typhimurium, Salmonella Gallinarum, or Salmonella Pullorum, including salmonellosis, Salmonella food poisoning, Fowl Typhoid and Pullorum disease, as well as for the control of Salmonella Enteritidis, Salmonella Typhimurium, Salmonella Gallinarum, and Salmonella Pullorum.

### DESCRIPTION OF DRAWINGS

FIG. 1 is an electron microscopy photograph of ΦCJ5, showing that ΦCJ5 belongs to the morphotype group of the family Siphoviridae, characterized by an isometric capsid and a long non-contractile tail;

FIG. 2 is of photographs showing the formation of ΦCJ5 plaques in a lawn of salmonella bacteria: A: in a lawn of SE; B: in a lawn of ST; C: in a lawn of SG; D: in a lawn of SP; E: in a lawn of SA; F: in a lawn of SB; G: in a lawn of SC; H: in a lawn of SD. Plaques formed in lawns of SE, ST, SG and SP, but not in lawns of SA, SB, SC and SD.

FIG. 3 is the result of SDS-PAGE of the isolated bacteriophage ΦCJ5, in which protein patterns of the bacteriophage are shown, with the appearance of major proteins at 38 kDa, 63 kDa, 52 kDa and 12 kDa (See-blue plus 2 prestained-standard (Invitrogen) used as marker);

FIG. 4 is the result of PFGE of the isolated bacteriophage ΦCJ5, showing the total genome size of approximately 42 kbp, with 5 kbp-DNA size standard (Bio-rad) serving as a size marker;

FIG. 5 is the result of PCR, performed using each primer set for the ΦCJ5 genomic DNA: A: a primer set of SEQ ID NOS. 8 and 9; B: a primer set of SEQ ID NOS. 10 and 11; C: a primer set of SEQ ID NOS. 12 and 13; D: a primer set of SEQ ID NOS. 14 and 15; E: a primer set of SEQ ID NOS. 16 and 17; F: a primer set of SEQ ID NOS. 18 and 19; and G: a primer set of SEQ ID NOS. 20 and 21. All of the PCR products were 500 by ˜3 kbp long;

FIG. 6 is the result of acid-resistance assay on the bacteriophage ΦCJ5, showing the number of surviving bacteriophage at pH 2.1, 2.5, 3.0, 3.5, 4.0, 5.5, 6.4, 6.9, 7.4, 8.0, 9.0, 9.8 and 11.0. The bacteriophage ΦCJ5 did not lose its activity until pH 3.0, but completely lost its activity at pH 2.5 or lower, as compared to control;

FIG. 7 is the result of heat-resistance assay on the bacteriophage ΦCJ5, showing the number of surviving bacteriophage at 37, 45, 53, 60, 70 and 80° C. for 0, 10, 30, 60 and 120 min. The bacteriophage ΦCJ5 maintained its activity at 70° C. for up to 2 hrs and 80° C. for 10 min, but completely lost its activity at 80° C. or higher for longer than 10 min;

FIG. 8 is the result of desiccation resistance assay on the bacteriophage ΦCJ5, performed with the aid of a Speed-Vac Concentration 5301 (Effendorf), in which when titer changes under the dry condition were measured in comparison with pre-drying titers, the activity was reduced by approximately 3×10 fold.

One embodiment of the present invention relates to a novel isolated bacteriophage having a specific bactericidal activity against Salmonella Enteritidis, Salmonella Typhimurium, Salmonella Gallinarum, or Salmonella Pullorum.

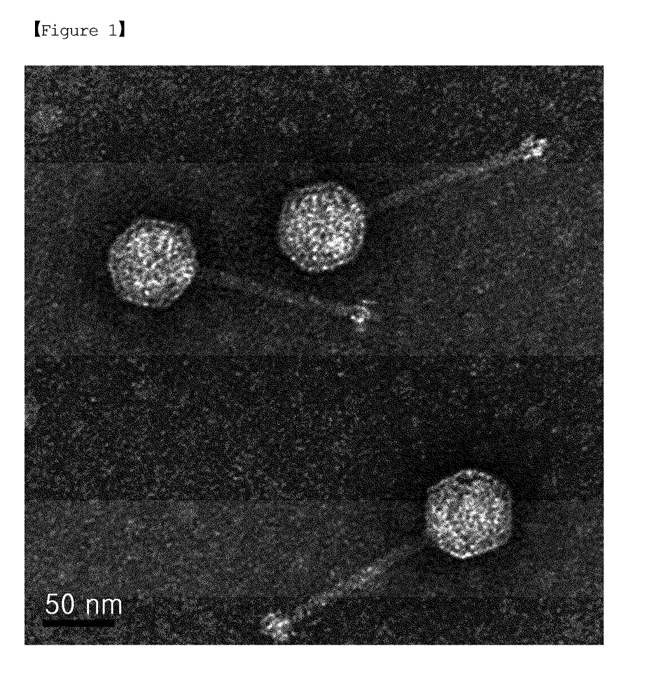

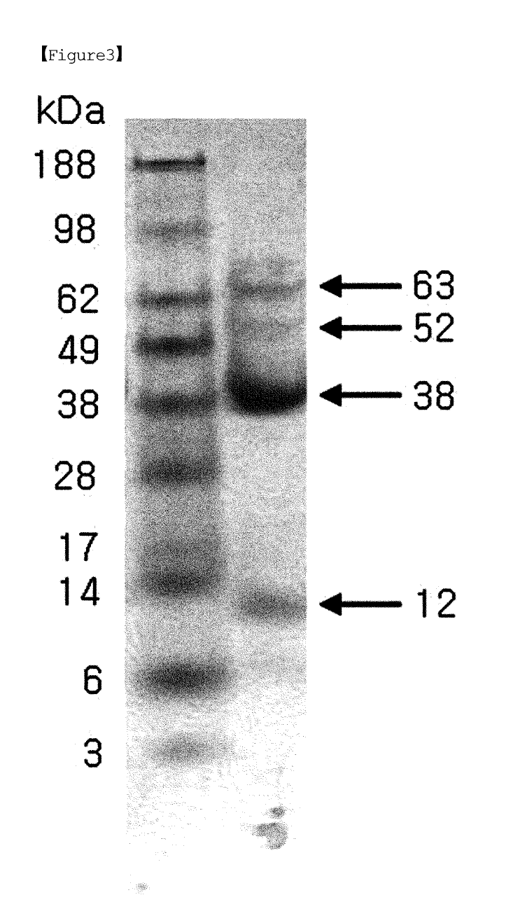

The bacteriophage according to some embodiments of the present invention belongs to the Siphoviridae family of morphotype B1 with the morphological structure consisting of an isometric capsid and a long, non-contractile tail, characterized by a total genome size of 41-43 kbp and major structural proteins ranging in size from 37 to 40 kDa, from 62 to 65 kDa, from 51 to 54 kDa and from 11 to 13 kDa (see FIGS. 1 and 3).

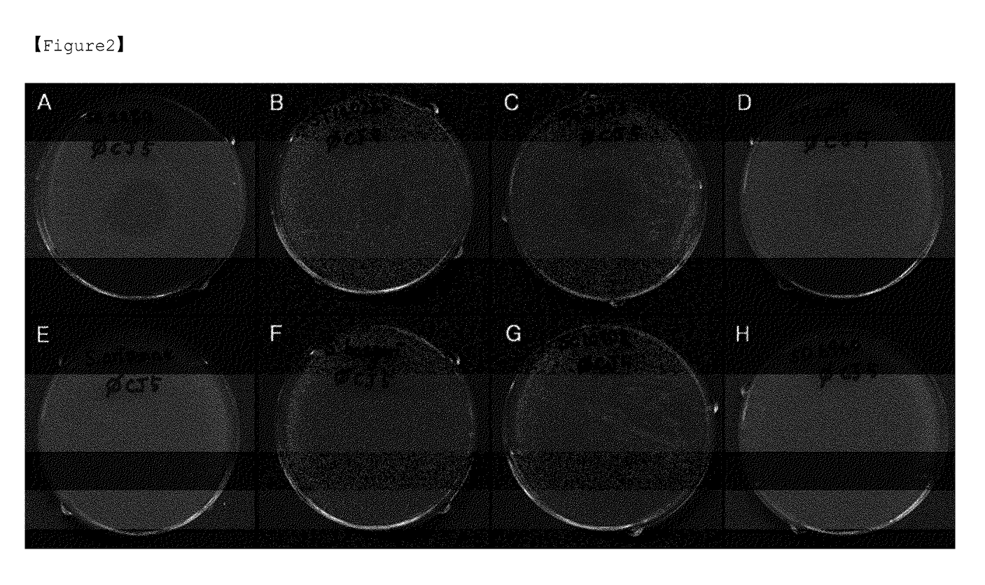

In a preferred embodiment, the bacteriophage according to some embodiments of the present invention shows the species specificity of specifically infecting only Salmonella Enteritidis, Salmonella Typhimurium, Salmonella Gallinarum, or Salmonella Pullorum, and thus do not have influence on the other species (see FIG. 2).

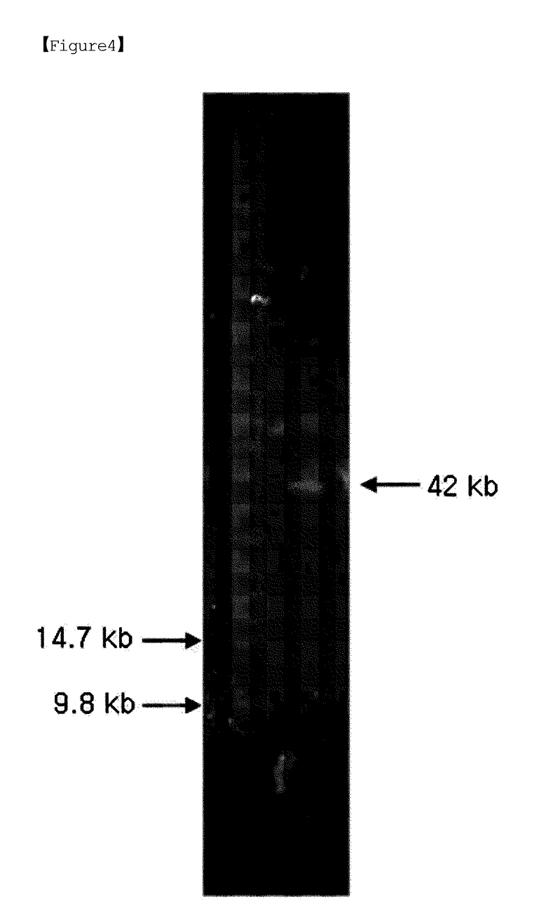

In a preferred embodiment, the bacteriophage according to some embodiments of the present invention has a total genome size of approximately 41-43 kbp, and preferably approximately 42 kbp (see FIG. 4). Further, the bacteriophage according to some embodiments may contain as parts of the genome thereof one or more nucleic acid molecules selected from the group consisting of SEQ ID NOS. 1 to 7. Preferably, the bacteriophage according to some embodiments may contain as parts of the genome thereof the nucleic acid molecules of SEQ ID NOS. 1 to 7.

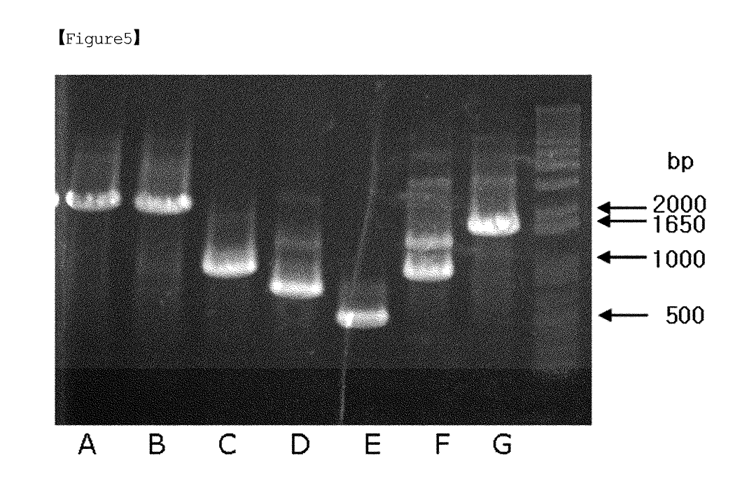

When PCR is performed in the presence of a primer set selected from among SEQ ID NOS. 8 and 9, SEQ ID NOS. 10 and 11, SEQ ID NOS. 12 and 13, SEQ ID NOS. 14 and 15, SEQ ID NOS. 16 and 17, SEQ ID NOS. 18 and 19, and SEQ ID NOS. 20 and 21, with the genome of the bacteriophage according to some embodiments of the present invention serving as a template, each PCR product is 500 by ˜3 kbp long (see FIG. 5). Preferably, When PCR is performed in the presence of all said primer sets, each each PCR product is 500 by ˜3 kbp long.

The term “nucleic acid molecule”, as used herein, is intended to include DNA (gDNA and cDNA) and RNA molecules. The term “nucleotides”, which when joined together, make up the structural units of nucleic acid molecules, encompass natural ones and sugar- or base-modified analogues thereof.

The bacteriophage according to some embodiments of the present invention has major structural proteins ranging in size from 37 to 40 kDa, from 62 to 65 kDa, from 51 to 54 kDa and from 11 to 13 kDa, and preferably corresponding to respective sizes of approximately 38 kDa, 63 kDa, 52 kDa and 12 kDa.

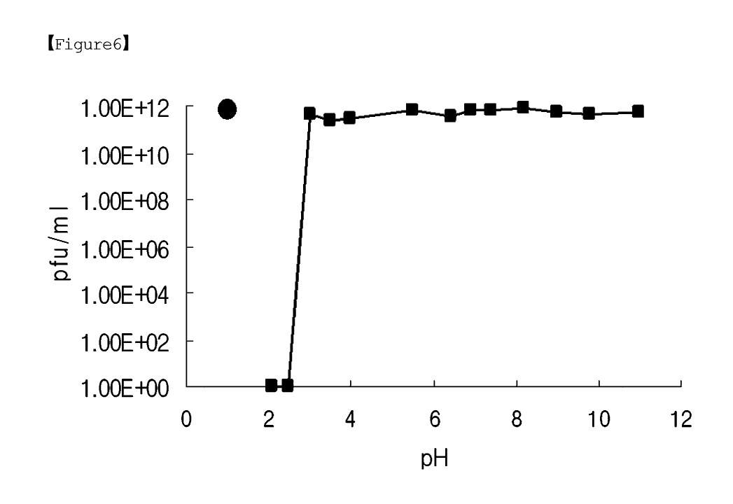

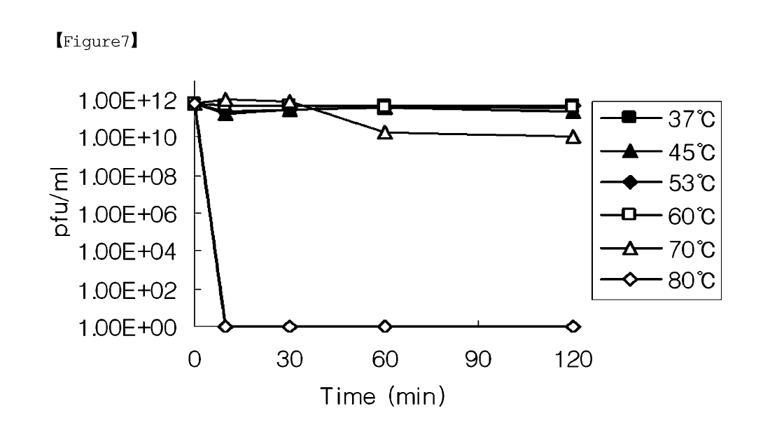

Further, the bacteriophage according to some embodiments of the present invention shows biochemical properties of being resistant to acid, heat and desiccation. In greater detail, the bacteriophage according to some embodiments of the present invention has excellent resistance to acid and heat so that it can survive over a wide pH range of from 3.0 to 11.0 and a heat range of from 37° C. to 70° C. (see FIGS. 6 and 7). With regard to the desiccation tolerance thereof, the bacteriophage can remain viable even under a high temperature and dry condition. Thanks to the superiority thereof in resistance to acid, heat and desiccation, the bacteriophage according to some embodiments of the present invention can be used in a wide range of temperature and pH, finding applications in compositions and products for the prevention and treatment of livestock diseases and livestock-mediated human diseases.

According to some embodiments, a bacteriophage was isolated from a sewage sample of a chicken slaughterhouse that bacteriophage was identified as having a specific bactericidal activity against SE, ST, SG and SP and the above characteristics, and was designated as Bacteriophage ΦCJ5 and deposited with the Korean Culture Center of Microorganisms (361-221, Honje 1, Seodaemun, Seoul) on Aug. 14, 2009 under accession number KCCM11028P.

In accordance with an example of the present invention, sewage samples were collected from chicken slaughterhouses and used to isolate there from bacteriophages that can lyse the host cell SE. They were also found to lyse SG, SP and ST (FIG. 2 and Table 1). An morphological examination under an electron microscope confirmed that the bacteriophage (ΦCJ5) belongs to the family Siphoviridae of morphotype B1 (FIG. 1).

According to some embodiments, a bacteriophage ΦCJ5 was found to have structural proteins of approximately 38 kDa, 63 kDa, 52 kDa and 12 kDa, as measured by a protein pattern analysis (FIG. 3).

Further, a genome analysis showed that ΦCJ5 has a total genome size of approximately 42 kbp (FIG. 4), with the nucleic acid molecules of SEQ ID NOS. 1 to 7 incorporated thereinto (Example 6). Also, the bacteriophage was found to be of very low genetic similarity with known bacteriophages as measured by the comparison of genetic similarity with other species, indicating that the bacteriophage is a novel one (Table 2). More particularly, when PCR was performed using the primer sets SEQ ID NOS. 8 and 9, SEQ ID NOS. 10 and 11, SEQ ID NOS. 12 and 13, SEQ ID NOS. 14 and 15, SEQ ID NOS. 16 and 17, SEQ ID NOS. 18 and 19, and SEQ ID NOS. 20 and 21, which were designed for ΦCJ5, the resulting PCR products were 500 by ˜3 kbp in size (FIG. 5).

Also, the phage plaques (clear zones formed in a lawn of cells on soft agar due to lysis by phage) resulting from the infection of ΦCJ5 into SE, ST, SG and SP were observed to have the same size and turbidity (FIG. 2).

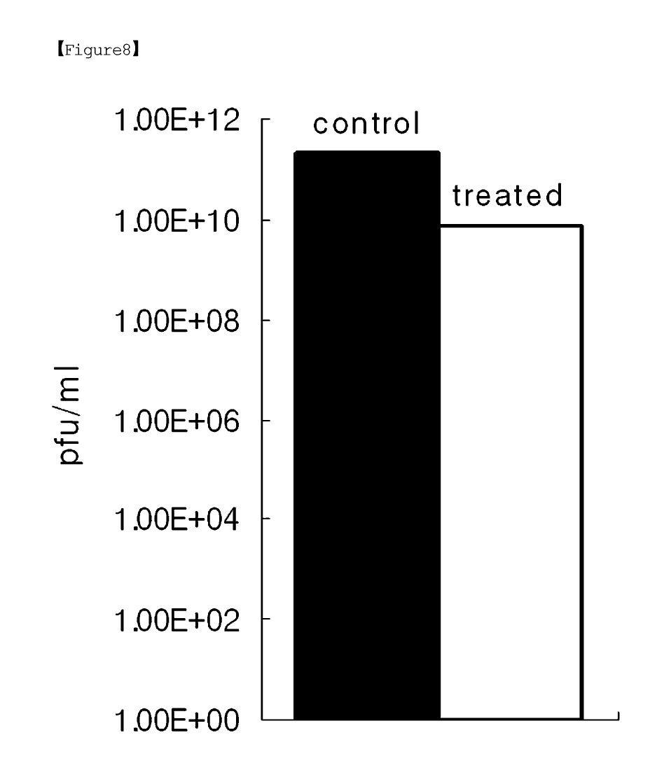

ΦCJ5 was examined for stability under a wide spectrum of pH and temperature. The bacteriophage was observed to survive over a pH range of from 3.0 to 11.0 (FIG. 6) and a temperature range of from 37° C. to 70° C. (FIG. 7) in addition to remaining stably viable even after desiccation at high temperature (120 min/60° C.) (FIG. 8). Also, the wild-type strains SE, ST, SG and SP were also found to fall within the host cell range of ΦCJ5 (Table 3). These data imply that the bacteriophage ΦCJ5 and its mutants can be applied to various products for the control of salmonella bacteria.

In accordance with another aspect thereof, the present invention pertains to a composition for the prevention or treatment of infectious diseases caused by one or more Salmonella bacteria selected from the group consisting of Salmonella enteritidis, Salmonella Typhimurium, Salmonella Gallinarum, and Salmonella Pullorum, comprising the bacteriophage as an active ingredient.

In a preferred embodiment, the composition may contain an antibiotic.

Having specific bactericidal activity against Salmonella enteritidis, Salmonella Typhimurium, Salmonella Gallinarum, and Salmonella Pullorum, according to some embodiments, the bacteriophage may be used for the purpose of preventing or treating the diseases caused by the bacteria. Preferably, examples of the infectious diseases include salmonellosis and Salmonella food poisoning by Salmonella enteritidis or Salmonella Typhimurium, Fowl Typhoid by Salmonella Gallinarum and Pullorum disease by Salmonella Pullorum include, but are not limited thereto.

As used herein, the term “salmonellosis” refers to symptoms following salmonella infection, such as fever, headache, diarrhea, and vomiting. That is, salmonellosis is an infection with bacteria of the genus Salmonella, with the accompaniment of two representative symptoms: septicemia such as typhoid fever; and acute gastroenteritis such as food poisoning, enteritis, and acute bacteremia.

As used herein, the term “prevention” is intended to encompass all actions for restraining or delaying disease progress through the administration of the composition. The term “treatment” in this context encompasses all actions for improving or beneficially changing the patient's condition through the administration of the composition.

According to some embodiments, the composition comprises ΦCJ5 in an amount of from 5×10² to 5×10¹² pfu/ml, and preferably in an amount of from 1×10⁶ to 1×10¹⁰ pfu/ml.

The composition according to some embodiments of the present invention may further comprise a pharmaceutically acceptable vehicle, and may be formulated together with the carrier into foods, medicines, and feed additives.

As used herein, the term “pharmaceutically acceptable vehicle” refers to a carrier or diluent that neither causes significant irritation to an organism nor degrades the biological activity and properties of the administered active ingredient. For use in the formulation of the composition into a liquid preparation, a pharmaceutically acceptable vehicle must be suitable for sterilization and biocompatibility. Examples include saline, sterile water, Ringer's solution, buffered physiological saline, albumin infusion solution, dextrose solution, maltodextrin solution, glycerol, and ethanol. They may be used alone or in any combination thereof. If necessary, another conventional additive, such as antioxidants, buffers, bacteriostatic agents, etc., may be added to the composition. When combined additionally with diluents, dispersants, surfactants, binders and/and lubricants, the composition according to some embodiments of the present invention may be formulated into injections such as aqueous solutions, suspensions and emulsions, or pills, capsules, granules, or tablets.

The prophylactic or therapeutic compositions according to some embodiments of the present invention may be locally applied to afflicted areas by coating or spraying. Alternatively, the composition according to some embodiments of the present invention may be administered through oral or parenteral routes. The parenteral routes are available for intravenous, intraperitoneal, intramuscular, subcutaneous or topical administration

Depending on a variety of factors including formulations, the mode of administration, the age, weight, sex, condition and diet of the patient or animal being treated, the time of administration, the route of administration, the rate of excretion, and reaction sensitivity, the suitable dosage of the composition according to some embodiments of the present invention will vary when it is applied, sprayed or administered. It will be apparent to those skilled in the art that when the pharmaceutical composition is administered to patients, the suitable total daily dose may be determined by an attending physician or veterinarian within the scope of sound medical judgment.

Oral dosage preparations of the composition according to some embodiments of the present invention may take the form of tablets, troches, lozenges, aqueous or emulsive suspensions, powders or granules, emulsions, hard or soft capsules, syrups, or elixirs. The oral dosage forms such as tablets and capsules may comprise a binder such as lactose, saccharose, sorbitol, mannitol, starch, amylopectin, cellulose or gelatin, an excipient such as dicalcium phosphate, a disintegrant such as corn starch or sweet potato starch, a lubricant such as magnesium stearate, calcium stearate, sodium stearylfumarate, or polyethylene glycol wax. For capsules, a liquid vehicle such as lipid may be further used.

For non-oral administration, the composition according to some embodiments of the present invention may be formulated into injections via subcutaneous, intravenous, or intramuscular routes, suppositories, or sprays inhalable via the respiratory tract, such as aerosols. Injection forms may be prepared by dissolving or suspending the composition according to some embodiments of the present invention, together with a stabilizer or a buffer, in water and loading the solution or suspension onto ampules or vial unit forms. For sprays, such as aerosols, a propellant for spraying a water-dispersed concentrate or wetting powder may be used in combination with an additive.

The “antibiotic”, as used herein, refer to a substance or compound that can be administered to animals to kill bacteria or inhibit their growth and is intended to encompass antiseptics, bactericidal agents and antibacterial agents. The animals are mammals, preferably mammals excluding humans. Thanks to the advantage of being of higher specificity for Salmonella over conventional antibiotics, the bacteriophage according to some embodiments of the present invention can kill the specific pathogens without affecting beneficial bacteria. Furthermore, the bacteriophage according to some embodiments of the present invention does not induce drug resistance so that it can be provided as a novel antibiotic with a long life cycle.

In accordance with a further aspect thereof, the present invention pertains to an animal feed or drinking water, comprising the bacteriophage as an active ingredient.

Feed additive antibiotics used in the fishery and livestock industry are intended to prevent infections. However, most of the currently available feed additive antibiotics are problematic in that they are apt to induce the occurrence of resistant strains and may be transferred to humans as they remain in livestock products. The uptake of such residual antibiotics may make human pathogens resistant to antibiotics, resulting in the spread of diseases. Furthermore, many kinds of feed additive antibiotics, usually used in combination in animal feeds, may cause the emergence of multidrug-resistant strains. Therefore, the bacteriophage according to some embodiments of the present invention can be used as a feed additive antibiotic that is eco-friendly enough to be a solution to the problems.

The animal feed according to embodiments the present invention may be prepared by adding the bacteriophage directly or in a separate feed additive form to an animal feed. In an animal feed, the bacteriophage according to some embodiments of the present invention may take a liquid or a dry form, and preferably exist as a dried powder. In this regard, the bacteriophage according to some embodiments of the present invention may be dried by air drying, natural drying, spray drying or freeze-drying, but these drying processes do not limit the present invention. The bacteriophage according to some embodiments of the present invention may be added as powder in an amount of from 0.05 to 10% by weight, preferably in an amount of from 0.1 to 2% by weight, based on the total weight of animal feed. The animal feed may comprise other conventional additives useful for the preservation thereof for a long term, in addition to the bacteriophage according to some embodiments of the present invention.

To the feed additive according to some embodiments of the present invention may be added another non-pathogenic microorganism.

The available additional microorganism may be selected from the group consisting of Bacillus subtilis that can produce protease, lipase and invertase, Lactobacillus sp. strain that can exert physiological activity and a function of decomposing under anaerobic conditions, such as in the stomach of cattle, filamentous fungi including Aspergillus oryzae (J Animal Sci 43: 910-926, 1976) that increases the weight of domestic animals, enhances milk production and helps the digestion and absorptiveness of feeds, and yeast including Saccharomyces cerevisiae (J Anim Sci 56:735-739, 1983).

The animal feed comprising ΦCJ5 in accordance with some embodiments of the present invention may include plant-based feeds, such as grains, nuts, food byproducts, seaweed, fiber, drug byproducts, oil, starches, meal, and grain byproducts, and animal-based feeds such as proteins, minerals, fat, single cell proteins, zooplankton, and food wastes, but is not limited thereto.

The feed additive comprising ΦCJ5 in accordance with some embodiments of the present invention may include additives for preventing quality deterioration, such as binders, emulsifiers and preservatives, and additives for increasing utility, such as amino acids, vitamins, enzymes, probiotics, flavorings, non-protein nitrogen, silicates, buffering agents, coloring agents, extracts, and oligosaccharides, but is not limited thereto.

When supplied with drinking water containing the bacteriophage according to some embodiments of the present invention, livestock can be continuously reduced in the population of Salmonella bacteria in the intestine thereof livestock. As a result, Salmonella-free livestock can be produced.

In accordance with still a further aspect thereof, the present invention pertains to a cleaner or a sanitizer, comprising the bacteriophage as an active ingredient.

The sanitizer comprising the bacteriophage as an active ingredient is very useful for food hygiene against, for example, food poisoning. In detail, the sanitizer may be utilized not only as an agent or a food additive for preventing salmonella contamination, but also in the production of salmonella-free livestock. In order to remove Salmonella, the sanitizer can also be sprayed over domestic sewages and applied to poultry barns, slaughterhouses, spots where livestock died, cooking spaces and cooking facilities, and any area where poultry acts.

Further, the cleaner comprising the bacteriophage as an active ingredient can be used on a body area of living animals, such as skin, feathers and the like, which is already or potentially contaminated with Salmonella bacteria.

In accordance with still another aspect, the present invention pertains to a method for the prevention or treatment of Salmonella Enteritidis- Salmonella Typhimurium-, Salmonella Gallinarum-, or Salmonella Pullorum-mediated infectious diseases, using a bacteriophage having a specific bactericidal activity against Salmonella Enteritidis, Salmonella Typhimurium, Salmonella Gallinarum, or Salmonella Pullorum.

In accordance with yet another aspect thereof, the present invention pertains to a method for the prevention or treatment of Salmonella Enteritidis- Salmonella Typhimurium-, Salmonella Gallinarum-, or Salmonella Pullorum-mediated infectious diseases, comprising administering to an animal in need thereof a composition for the prevention or treatment of Salmonella Enteritidis-, Salmonella Typhimurium-, Salmonella Gallinarum-, or Salmonella Pullorum-mediated diseases.

The composition according to some embodiments of the present invention may be administered in the form of a pharmaceutical formulation into animals or may be ingested as a mixture with animal feed or drinking water by animals and preferably as a mixture with animal feed. In the present invention, the animals include cattle, pigs, chicken, poultry and humans, but are not limited thereto.

As long as it reaches target tissues, any route, whether oral or parenteral, may be taken for administering the composition according to some embodiments of the present invention. In detail, the composition according to some embodiments of the present invention may be administered via oral, rectal, topical, intravenous, intraperitoneal, intramuscular, intraarterial, transdermal, intranasal, and inhalation routes.

The method for the treatment of diseases in accordance with some embodiments of the present invention comprises administering the composition according to some embodiments of the present invention in a therapeutically effective amount. It is apparent to those skilled in the art that the total daily dose should be determined by an attending physician or veterinarian within the scope of sound medical judgment. The therapeutically effective amount for a given patient may vary depending on various factors well known in the medical art, including the kind and degree of the response to be achieved, the patient's age, body weight, state of health, sex, and diet, time and route of administration, the secretion rate of the composition, the time period of therapy, concrete compositions according to whether other agents are used therewith or not, etc.

A better understanding of some embodiments of the present invention may be obtained through the following examples which are set forth to illustrate, but are not to be construed as limiting the present invention.

### Example 1

### Salmonella Bacteriophage Isolation

1-1. Bacteriophage Screening and Single Bacteriophage Isolation

50 ml of each sample from a chicken slaughterhouse and a nearby sewage disposal plant was transferred to a centrifuge tube, and centrifuged at 4000 rpm for 10 min, followed by filtering the supernatant through a 0.45 μm filter. 18 mL of the sample filtrate was mixed with 150 μl of a Salmonella Enteritidis (hereinafter referred to as “SE”) shaking culture medium (OD₆₀₀=2) and 2 ml of 10× Luria-Bertani medium (hereinafter referred to as “LB medium”, tryptone 10 g; yeast extract 5 g; NaCl 10 g; in a final volume of 1 L). The mixture was cultured at 37° C. for 18 hrs and then centrifuged at 4000 rpm for 10 min after which the supernatant was filtered through a 0.45 μm filter. Separately, a mixture of 3 ml of 0.7% agar (w/v) and 150 μl of the SE shaking culture medium (OD₆₀₀=2) was poured across an LB plate and allowed to solidify. Over this plate was spread 10 μl of the culture filtrate, followed by incubation for 18 hrs at 37° C. (0.7% agar was used as “top-agar” and the titration of phage lysate was performed on the top-agar, called soft agar overlay technique).

A dilution of the sample culture medium containing the phage lysate was mixed with 150 μL of an SE shaking culture medium (OD₆₀₀=2) and subjected to soft agar overlay assay to produce single plaques. Since a single plaque consisted of the same bacteriophage, one plaque was taken and dissolved in 400 μL of an SM solution (NaCl, 5.8 g; MgSO₄7H₂O, 2 g; 1 M Tris-Cl (pH7.5), 50 ml; H₂O, in a final volume of L), and left for 4 hrs at room temperature to isolate single bacteriophages. To amplify the isolated bacteriophage, 100 μL of the supernatant was taken from the single bacteriophage solution, mixed with 12 mL of 0.7% agar and 500 μL of an SE shaking culture medium, and subjected to a soft agar overlay assay on an LB plate (150 mm in diameter). 15 mL of an SM solution was poured to a plate in which lysis had been completed, after which the plate was gently shaken for 4 hrs at room temperature to elute the bacteriophages from the top-agar. The SM solution containing the eluted bacteriophages was recovered, and chloroform was added in an amount corresponding to 1% of the final volume, and mixed well for 10 min. After centrifugation at 4000 rpm for 10 minutes, the resulting supernatant was filtered through a 0.45 μm filter, and stored in the refrigerator until use.

1-2. Large-Scale Batches of Bacteriophage

The selected bacteriophage was cultured at a large scale using SE. SE was cultured with shaking. After an aliquot of 1.5×10¹⁰ cfu (colony forming units) was centrifuged at 4000 rpm for 10 min, the pellet was re-suspended in 4 ml of an SM solution. Into the suspension was inoculated 7.5×10⁷ pfu (plaque forming unit) of the bacteriophage at an MOI (multiplicity of infection) of 0.005), followed by incubation at 37° C. for 20 min. This solution was inoculated into 150 mL of an LB media in a flask, and cultured at 37° C. for 5 hrs. Chloroform was added in an amount corresponding to 1% of the final volume before the culture solution was shaken for 20 min. DNase I and RNase A were added to a final concentration of 1 μg/ml, each. The solution was left at 37° C. for 30 min. NaCl and PEG (polyethylene glycol) were added to a final concentration of 1 M and 10% (w/v), respectively and left at 4° C. for an additional 3 hrs. The solution was centrifuged at 4° C. and 12,000 rpm for 20 min to discard the supernatant. A suspension of the pellet in 5 mL of an SM solution was left at room temperature for 20 minutes and mixed well with 4 mL of chloroform. After centrifugation at 4° C. and 4000 rpm for 20 min, the supernatant was filtered through a 0.2 μm filter and then subjected to ultracentrifugation using a glycerol density gradient to purify ΦCJ5 (density: 40%, 5% glycerol at 35,000 rpm and 4° C. for 1 hr). The purified ΦCJ5 was re-suspended in 300 μL of an SM solution, followed by titration. ΦCJ5 was deposited with the Korean Culture Center of Microorganisms (361-221, Honje 1, Seodaemun, Seoul) on Aug. 14, 2009 under accession number KCCM11028P.

### Example 2

### Examination on φCJ5 Infection of Salmonella

To analyze the selected bacteriophage for lytic activity on Salmonella species other than SE, attempts were made of cross infection with other Salmonella species. As a result, ΦCJ5 did not infect SC (Salmonella Choleraesuis), SD (Salmonella Derby), SA (Salmonella arizonae), and SB (Salmonella bongori), but infected SE, ST (Salmonella typhimurium), SG (Salmonella Gallinarum) and SP (Salmonella Pullorum) (see Example 11). The results are summarized in Table 1, below and shown in FIG. 2.

TABLE 1
ΦCJ5 Infection of Salmonella

| Sero | Strain | Plaque | Sero | Strain | Plaque |
|---|---|---|---|---|---|
| type | name | formation | type | name | formation |
| SE | SGSC 2282 | ◯ | SA | ATCC 13314 | X |
| ST | ATCC 14028 | ◯ | SB | ATCC 43975 | X |
| SG | SGSC 2293 | ◯ | SC | ATCC 10708 | X |
| SP | SGSC 2295 | ◯ | SD | ATCC 6960 | X |

ATCC: The Global Bioresource Center
SGSC: Salmonella Genetic Stock Center

### Example 3

### Morphology of Bacteriophage ΦCJ5

The purified ΦCJ5 was diluted in a 0.01% gelatin solution, and then fixed in a 2.5% glutaraldehyde solution. The sample was dropped onto a carbon-coated mica plate (ca.2.5×2.5 mm), adapted for 10 min, and washed with sterile distilled water. A carbon film was mounted on a copper grid, stained with 4% uranyl acetate for 30-60 sec, and dried. Examination under a JEM-1011 transmission electron microscope (at 80 kV, magnification of X 120,000˜X 200,000), as shown in FIG. 1, had it that the purified ΦCJ5 consisted morphologically of an isometric capsid and a long non-contractile tail, indicating that it belongs to the family Siphoviridae of morphotype B1.

### Example 4

### Protein Pattern Analysis of ΦCJ5

15 μL of a ΦCJ5 solution purified at a titer of 10¹² pfu/ml was mixed with 3 μL of a 5× SDS sample solution, and heated for 5 min. The total protein of ΦCJ5 was run on 4˜12% NuPAGE Bis-Tris gel (Invitrogen). Then, the gel was stained with Coomassie blue for 1 hr at room temperature. Major bands were detected at approximately 38 kDa, 63 kDa, 52 kDa and 12 kDa, as shown in FIG. 3.

### Example 5

### Total Genomic DNA Size of ΦCJ5

Genomic DNA of ΦCJ5 was isolated using ultracentrifugation. In this regard, to a purified ΦCJ5 culture medium were added EDTA (ethylenediaminetetraacetic acid (pH8.0)), proteinase K, and SDS (sodium dodecyl sulfate) at a final concentration of 20 mM, 50 ug/ml, and 0.5% (w/v), respectively, followed by incubation at 50° C. for 1 hr. An equal volume of phenol (pH 8.0) was added and mixed well. After centrifugation at 12,000 rpm and room temperature for 10 min, the supernatant was mixed well with an equal volume of PC (phenol:chloroform=1:1). Another centrifugation at 12,000 rpm and room temperature for 10 min produced a supernatant which was then mixed with 1/10 volume of 3 M sodium acetate and two volumes of cold 95% ethanol, and left at −20° C. for 1 hr. After centrifugation at 0° C. and 12,000 rpm for 10 min, the supernatant was completely removed, and the DNA pellet was dissolved in 50 μL of TE (Tris-EDTA (pH 8.0)). The extracted DNA was diluted 10-fold, and measured for absorbance at OD₂₆₀ to determine its concentration 1 μg of the total genomic DNA was loaded onto 1% PFGE (pulse-field gel electrophoresis) agarose gel and electrophoresed at room temperature for 20 hrs with the aid of a BIORAD PFGE system program 7 (size range 25-100 kbp; switch time ramp 0.4-2.0 seconds, linear shape; forward voltage 180 V; reverse voltage 120 V). As shown in FIG. 4, the genomic DNA of ΦCJ5 was approximately 42 kb long.

### Example 6

### Genetic Analysis of ΦCJ5

The genetic analysis of the purified ΦCJ5 started with double digesting 5 μg of the genomic DNA of ΦCJ5 with the restriction enzymes EcoRV, ScaI and HinC II. A T-blunt vector (Sogent) was employed. The digested genomic DNA was mixed at a ratio of 3:1 with the vector, and ligated at 16° C. for 2 hrs. The resulting recombinant vector was transformed into E. coli DH5α which was then plated on an LB plate containing kanamycin and X-gal (5-bromo-4-chloro-3-indolyl-beta-D-galactopyranoside) for blue/white selection. The selected colonies were cultured for 16 hrs in a culture medium containing the antibiotic with shaking. Then, plasmids were extracted using a plasmid purification kit (Promega).

The cloning of the plasmids was confirmed by PCR using a primer set of M13 forward and M13 reverse, and selection was made only of insert fragments having a size of 1 kb or longer. Their base sequences were analyzed using the primer sets. The base sequences thus obtained were given in SEQ ID NOS. 1 to 7, respectively, each being 500 by ˜3 kbp long, and analyzed for sequence similarity with the aid of NCBI blastx and blstn programs, and the results are summarized in Table 2, below.

As is apparent from the data of Table 2, ΦCJ5 has a protein similarity of from 76% to 94% with hypothetical protein, tailspike protein and p21 protein of Salmonella phage KS7, salmonella phage SETP3, and bacteriophage MB78.

However, a low similarity in nucleotide sequence was found therebetween, as analyzed by the NCBI blastn program, which thus indicates, along with the low query coverage, that ΦCJ5 is a novel bacteriophage species.

TABLE 2
Sequence Similarity between ΦCJ5 and Other Bacteriophages

| | | | Blastx | | | |
|---|---|---|---|---|---|---|
| No | Organism | Protein | Query | Subject | Identity | e-value |
| 1 | Salmonella phage KS7 | hypothetical | 108-1088 | 165-494 | 292/330 | 2e−143 |
| | | protein | | | (88%) | |
| | Salmonella phage KS7 | hypothetical | 665-168 | 1-166 | 162/166 | 1e−93 |
| | | protein | | | (97%) | |
| 2 | Salmonella phage KS7 | hypothetical | 537-836 | 1-100 | 100/100 | 9e−53 |
| | | protein | | | (100%) | |
| | Salmonella phage KS7 | hypothetical | 759-85 | 1-225 | 224/225 | 3e−128 |
| | | protein | | | (99%) | |
| | | BPKS7gp16 | | | | |
| | Salmonella phage | terminase | 759-85 | 1-225 | 221/225 | 1e−126 |
| | SETP7(11) | | | | (98%) | |
| 3 | Salmonella phage | hypothetical | 90-794 | 150-304 | 230/235 | 2e−125 |
| | SETP3 | protein | | | (97%) | |
| | Salmonella phage | hypothetical | 1035-385 | 167-384 | 199/218 | 3e−102 |
| | SETP3 | protein | | | (91%) | |
| 4 | Salmonella phage KS7 | hypothetical | 113-862 | 194-443 | 223/250 | 1e−119 |
| | | protein | | | (89%) | |
| | | BPKS7gp56 | | | | |
| | Salmonella phage KS7 | hypothetical | 835-83 | 193-443 | 239/251 | 4e−119 |
| | | protein | | | (98%) | |
| 5 | Salmonella phage KS7 | hypothetical | 76-552 | 562-728 | 152/167 | 3e−80 |
| | | protein | | | (91%) | |
| | Bacteriophage MB78 | 22 kDa protein | 374-228 | 1-49 | 46/49 | 3e−27 |
| | | | | | (93%) | |
| | Salmonella phage KS7 | hypothetical | 576-133 | 581-728 | 143/148 | 8e−78 |
| | | protein | | | (96%) | |
| | Salmonella phage | hypothetical | 576-133 | 581-728 | 141/148 | 9e−77 |
| | SETP3 | protein | | | (95%) | |
| | Bacteriophage MB78 | 22 kDa protein | 311-457 | 1-49 | 45/49 | 3e−25 |
| | | | | | (91%) | |
| 6 | Salmonella phage | tail component | 109-1026 | 34-340 | 287/307 | 6e−153 |
| | SETP3 | protein | | | (93%) | |
| | Salmonella phage KS7 | hypothetical | 109-1026 | 145-451 | 284/307 | 6e−152 |
| | | protein | | | (92%) | |
| | Bacteriophage MB78 | putative 10 kDa | 673-1011 | 1-114 | 94/114 | 2e−42 |
| | | protein | | | (82%) | |
| | Salmonella phage | tail component | 940-83 | 56-341 | 265/286 | 3e−142 |
| | SETP3 | protein | | | (92%) | |
| | Salmonella phage KS7 | hypothetical | 940-83 | 167-452 | 263/286 | 3e−141 |
| | | protein | | | (91%) | |
| | Bacteriophage MB78 | putative 10 kDa | 442-101 | 1-114 | 107/114 | 6e−54 |
| | | protein | | | (93%) | |
| 7 | Salmonella phage | tailspike | 1130-258 | 394-684 | 273/291 | 1e−154 |
| | SETP13 | protein | | | (93%) | |
| | Salmonella phage KS7 | tailspike | 1130-258 | 152-442 | 268/291 | 1e−152 |
| | (KS5) | protein | | | (92%) | |
| | Enterobacteria phage | gp9 | 1130-258 | 373-665 | 216/293 | 1e−121 |
| | ST104 | | | | (73%) | |
| | Salmonella phage | tailspike | 78-728 | 180-401 | 210/222 | 1e−117 |
| | SETP13 | protein | | | (94%) | |

### Example 7

### Design of ΦCJ5-Specific Primer Sequences

In order to identify ΦCJ5, ΦCJ5-specific primers were designed on the basis of SEQ ID NOS. 1 to 7. PCR was performed using each primer set of SEQ ID NOS. 8 and 9, SEQ ID NOS. 10 and 11, SEQ ID NOS. 12 and 13, SEQ ID NOS. 14 and 15, SEQ ID NOS. 16 and 17, SEQ ID NOS. 18 and 19, and SEQ ID NOS. 20 and 21. 0.1 μg of the genomic DNA of bacteriophage and 0.5 pmol of each primer were added to a pre-mix (Bioneer), and the final volume was adjusted to 20 μL. PCR was performed with 30 cycles of denaturation; 94° C. 30 sec, annealing; 60° C. 30 sec, and polymerization; 72° C., 1 min. The PCR products thus obtained were approximately 500 by ˜3 kbp long, with the primer sets of SEQ ID NOS. 8 and 9, SEQ ID NOS. 10 and 11, SEQ ID NOS. 12 and 13, SEQ ID NOS. 14 and 15, SEQ ID NOS. 16 and 17, SEQ ID NOS. 18 and 19, and SEQ ID NOS. 20 and 21. The results are shown in FIG. 5.

### Example 8

### pH Stability of Bacteriophage

In order to determine whether ΦCJ5 survives the low pH environment in the stomach of chicken, ΦCJ5 was assayed for stability in a wide range of pH (pH 2.1, 2.5, 3.0, 3.5, 4.0, 5.5, 6.4, 6.9, 7.4, 8.2, 9.0, 9.8, and 11.0). Various pH solutions (sodium acetate buffer (pH 2.1, 4.0, pH 5.5, and pH 6.4), sodium citrate buffer (pH 2.5, pH 3.0, and pH 3.5), sodium phosphate buffer (pH 6.9 and pH 7.4) and Tris-HCl (pH 8.2, pH 9.0, pH 9.8 and pH 11.0)) were prepared to have a concentration of 0.2 M. 180 μL of each pH solution was mixed with 20 μL of a bacteriophage solution (1.0×10¹¹ pfu/ml) to give each pH solution a concentration of 1 M, followed by incubation at room temperature for 2 hr. The reaction solution was serially diluted, and 10 μL of each dilution was cultured at 37° C. for 18 hrs by a soft agar overlay method to determine the titers of the phage lysates. Titer changes with pH were measured to determine the stability of bactriophage over pH in comparison to titers of ΦCJ5 at 0 hr. The results showed that the bacteriophage did not lose its activity and remained stable down to pH 3.0. However, it lost its activity at pH 2.5 or lower. The results are shown in FIG. 6.

### Example 9

### Heat Stability of Bacteriophage

For use as a feed additive, the bacteriophage was assayed for stability to the heat generated during a formulation process. In this regard, 200 μL of a ΦCJ5 solution with a titer of 1.0×10¹¹ pfu/ml was incubated at 37° C., 45° C., 53° C., 60° C., 70° C., or 80° C. for 0 min, 10 min, 30 min, 60 min and 120 min. The solution was serially diluted, and 10 μL of each dilution was cultured at 37° C. for 18 hrs by a soft agar overlay method to determine the titers of phage lysates. Titer changes with temperature and exposure time were measured to determine the stability of bacteriophage to heat in comparison to titers at 0 min and 37° C. The results showed that the bacteriophage did not lose its activity at 70° C. for up to 2 hrs, but was deactivated at 80° C. The results are shown in FIG. 7.

### Example 10

### Desiccation Tolerance of Bacteriophage

For use as a feed additive, the bacteriophage was assayed for tolerance to the dry condition set for a formulation process. On the basis of the results obtained from the heat stability assay, a desiccation assay was performed at 60° C. for 120 min. Using a Speed-Vac Concentration 5301 (Eppendorf), 200 mL of a ΦCJ5 solution having a titer of 1.0×10¹¹ pfu/ml was dried. The powder thus obtained was re-suspended in 200 mL of an SM solution and measured for titer values. After desiccation, the bacteriophage was decreased in activity by approximately 3×10 fold, compared to pre-drying titers. The results are shown in FIG. 8.

### Example 11

### Spectrum of Wile-Type Host Cell Strains to which Bacteriophage Infects

ΦCJ5 was assayed for lytic activity against Korean wild-type Salmonella Enteritidis (36 strains), Salmonella Typhimurium (22 strains), Salmonella Gallinarum (56 strains), Salmonella Pullorum (19 strains), Salmonella Choleraesuis (2 strains), Salmonella Derby (4 strains) and Salmonella Arizona (1 strain), and Salmonella Bongori (1 strain), obtained from Laboratory of Avian Diseases, College of Veterinary Medicine, Seoul National University, and National Veterinary Research and Quarantine Service and the Korea Centers for Disease Control and Prevention, in addition to the strains used in at least some embodiments of the present invention, SE(SGSC 5E2282), ST(ATCC ST14028), SG(SGSC SG2293), and SP(SGSC SP2295). 150 μL of each strain shaking culture medium (OD₆₀₀=2) was mixed, and 10 μL of ΦCJ5 solution (10¹⁰ pfu/ml) was cultured at 37° C. for 18 hrs using a soft agar overlay method to monitor the formation of plaques. It was observed that the bacteriophage ΦCJ5 showed lytic activity of 91% against all the wild-type strains SE, ST, SG and SP. The results are summarized in Table 3, below.

TABLE 3
Lytic Activity of ΦCJ5 against Korean Wild-Type Strains SE, ST, SG,
and SP

| | | ΦCJ5 | | | ΦCJ5 |
|---|---|---|---|---|---|
| Sero | | Plaque | Sero | | Plaque |
| type | Strain name | formation | type | Strain name | formation |
| SG | SNU SG1 | ◯ | ST | SNU ST1 | X |
| | SNU SG2 | ◯ | | SNU ST2 | ◯ |
| | SNU SG3 | ◯ | | SNU ST3 | X |
| | SNU SG4 | ◯ | | SNU ST4 | ◯ |
| | SNU SG5 | ◯ | | SNU ST7 | ◯ |
| | SNU SG6 | ◯ | | SNU ST8 | ◯ |
| | SNU SG7 | ◯ | | SNU ST11 | ◯ |
| | SNU SG8 | ◯ | | SNU ST12 | ◯ |
| | SNU SG9 | ◯ | | SNU ST13 | X |
| | SNU SG10 | ◯ | | SNU ST14 | ◯ |
| | SNU SG11 | ◯ | | SNU ST17 | ◯ |
| | SNU SG12 | ◯ | | SNU ST18 | X |
| | SNU SG13 | ◯ | | SNU ST19 | X |
| | SNU SG14 | ◯ | | SNU ST20 | ◯ |
| | SNU SG15 | ◯ | | SNU ST25 | ◯ |
| | SNU SG16 | ◯ | | SNU ST26 | ◯ |
| | SNU SG17 | ◯ | | SNU ST37 | ◯ |
| | SNU SG18 | ◯ | | SNU ST38 | ◯ |
| | SNU SG19 | ◯ | | SNU ST41 | X |
| | SNU SG20 | ◯ | | SNU ST42 | ◯ |
| | SNU SG21 | ◯ | | ATCC UK1 | ◯ |
| | SNU SG22 | ◯ | | ATCC 14028S | ◯ |
| | SNU SG23 | ◯ | | SGSC STM1412 | ◯ |
| | SNU SG24 | ◯ | | SGSC STM260 | ◯ |
| | SNU SG25 | ◯ | | SGSC STM | ◯ |
| | | | | SA2197 | |
| | SNU SG26 | ◯ | SE | SGSC SE2282 | ◯ |
| | SNU SG27 | ◯ | | SGSC SE2377 | ◯ |
| | SNU SG28 | ◯ | | PT4 S1400194 | ◯ |
| | SNU SG30 | ◯ | | PT4 LA52 | ◯ |
| | SNU SG31 | ◯ | | NVRQS SE004 | ◯ |
| | SNU SG32 | ◯ | | NVRQS SE005 | ◯ |
| | SNU SG33 | ◯ | | KCDC SE008 | ◯ |
| | SNU SG34 | ◯ | | KCDC SE009 | ◯ |
| | SNU SG36 | ◯ | | KCDC SE010 | ◯ |
| | SNU SG37 | ◯ | | KCDC SE011 | ◯ |
| | SNU SG38 | ◯ | | KCDC SE012 | ◯ |
| | SNU SG39 | ◯ | | KCDC SE013 | ◯ |
| | SNU SG40 | ◯ | | KCDC SE014 | ◯ |
| | SNU SG41 | ◯ | | KCDC SE015 | ◯ |
| | SNU SG42 | ◯ | | KCDC SE016 | ◯ |
| | SNU SG43 | ◯ | | KCDC SE017 | ◯ |
| | SNU SG44 | ◯ | | KCDC SE018 | ◯ |
| | SNU SG45 | ◯ | | KCDC SE019 | ◯ |
| | SNU SG46 | ◯ | | KCDC SE020 | ◯ |
| | SNU SG47 | ◯ | | KCDC SE021 | ◯ |
| | SNU SG48 | ◯ | | KCDC SE022 | ◯ |
| | SNU SG49 | ◯ | | KCDC SE023 | ◯ |
| | SNU SG50 | ◯ | | KCDC SE024 | ◯ |
| | SGSC SG9184 | ◯ | | KCDC SE025 | ◯ |
| | SGSC SG2292 | ◯ | | KCDC SE026 | ◯ |
| | SGSC SG2293 | ◯ | | KCDC SE027 | ◯ |
| | SGSC SG2744 | ◯ | | KCDC SE028 | ◯ |
| | SGSC SG2796 | ◯ | | KCDC SE029 | ◯ |
| SP | SNU SP1 | ◯ | | KCDC SE030 | ◯ |
| | SNU SP4 | ◯ | | KCDC SE031 | ◯ |
| | SNU SP5 | ◯ | | KCDC SE032 | ◯ |
| | SNU SP8 | ◯ | | KCDC SE033 | ◯ |
| | SNU SP11 | ◯ | | KCDC SE034 | ◯ |
| | SGSC SP2294 | ◯ | | KCDC SE035 | ◯ |
| | SGSC SP2295 | ◯ | | KCDC SE036 | ◯ |
| | SGSC SP2737 | ◯ | | KCDC SE037 | ◯ |
| | SGSC SP2739 | ◯ | SC | ATCC SC10708 | X |
| | SGSC SP2742 | ◯ | | ATCC SC2929 | X |
| | SGSC SP2743 | ◯ | SD | ATCC SD6960 | X |
| | SGSC SP2745 | ◯ | | ATCC SD2466 | ◯ |
| | SGSC SP2751 | ◯ | | ATCC SD2467 | X |
| | SGSC SP4663 | ◯ | | ATCC SD2468 | X |
| | SGSC SP4664 | ◯ | SA | ATCC SA13314 | X |
| | SGSC SP4665 | ◯ | SB | ATCC SB43975 | X |
| | SGSC SP4666 | ◯ | | | |
| | SGSC SP4667 | ◯ | | | |
| | SGSC SA1684 | ◯ | | | |

SNU: Laboratory of Avian Diseases, College of Veterinary Medicine, Seoul National University
SGSC: salmonella genetic stock center
NVRQS: National Veterinary Research & Quarantine Service
KCDC: Korean Centers for Disease Control and prevention

### Example 12

### Toxicity Assay of Bacteriophage

For safety use in the prevention of salmonellosis, salmonella food poisoning, fowl typhoid and pullorum, the bacteriophage ΦCJ5 was in vivo assayed for toxicity using Rat. For this, single oral dosage toxicity, intravenous injection toxicity, and toxicity against internal normal bacteria were assayed. In the intravenous injection toxicity assay, toxicity was examined when the bacteriophage was present in blood. As for the assay for toxicity against internal normal bacteria, it was intended to examine the influence of the bacteriophage on representative internal normal bacteria. As a result, the novel bacteriophage ΦCJ5 was found to be non-toxic.

Having specific bactericidal activity against one or more Salmonella bacteria selected from the group consisting of Salmonella Enteritidis (SE), Salmonella Typhimurium (ST), Salmonella Gallinarum (SG), and Salmonella Pullorum (SP) without affecting beneficial bacteria, in addition to showing excellent tolerance to acid, heat and desiccation, as described hitherto, the novel bacteriophage according to some embodiments of the present invention can be widely used as an active ingredient for therapeutic agents, animal feeds or drinking water, cleaners and sanitizers for preventing and treating the infectious diseases caused by Salmonella Enteritidis, Salmonella Typhimurium, Salmonella Gallinarum or Salmonella Pullorum including salmonellosis, Salmonella food poisoning, Fowl Typhoid, and Pullorum disease.

Although preferred embodiments of the present invention have been disclosed for illustrative purposes, those skilled in the art will appreciate that various modifications, additions and substitutions are possible, without departing from the scope and spirit of the invention as disclosed in the accompanying claims.

1. An isolated bacteriophage, belonging to the family Siphoviridae of morphotype B 1, with a specific bactericidal activity against one or more Salmonella bacteria selected from the group consisting of Salmonella Enteritidis, Salmonella Typhimurium, Salmonella Gallinarum, and Salmonella Pullorum, characterized by one of the following properties:
1) the bacteriophage has a total genome size of 41˜43 kbp;
2) the bacteriophage contains as a part of the genome thereof at least one nucleic acid sequence selected from among SEQ ID NO. 1, SEQ ID NO. 2, SEQ ID NO. 3, SEQ ID NO. 4, SEQ ID NO. 5, SEQ ID NO. 6, and SEQ ID NO. 7; and
3) the bacteriophage has structural proteins ranging in size from 37-40 kDa, 62-65 kDa, 51-54 kDa and 11-13 kDa.

2. The bacteriophage according to claim 1, having a morphological structure composed of an isometric capsid and a long non-contractile tail.

3. The bacteriophage according to claim 1, where when PCR is performed in a presence of a primer set selected from among SEQ ID NOS. 8 and 9, SEQ ID NOS. 10 and 11, SEQ ID NOS. 12 and 13, SEQ ID NOS. 14 and 15, SEQ ID NOS. 16 and 17, SEQ ID NOS. 18 and 19, and SEQ ID NOS. 20 and 21, with the genome of the bacteriophage serving as a template, each PCR product is 500 by ˜3 kbp long.

4. The bacteriophage according to claim 1, showing at least one of the following properties;
1) tolerance to a range of from pH 3.0 to pH 11.0;
2) tolerance to a heat range of from 37° C. to 70° C.; and
3) tolerance to desiccation under a condition of 60° C./120 min.

5. The bacteriophage according to claim 1, being identified by accession number KCCM11028P.

6. A composition for prevention or treatment of infectious diseases caused by one or more Salmonella strains selected from the group consisting of Salmonella Enteritidis, Salmonella Typhimurium, Salmonella Gallinarum, and Salmonella Pullorum, comprising the bacteriophage of claim 1.

7. The composition according to claim 6, wherein the infectious diseases are salmonellosis and salmonella food poisoning when caused by Salmonella enteritidis or Salmonella Typhimurium, Fowl typhoid when caused by Salmonella Gallinarum and pullorum when caused by Salmonella Pullorum.

8. The composition according to claim 6, being used as an antibiotic.

9. An animal feed or drinking water, comprising the bacteriophage of claim 1.

10. A sanitizer and cleaner, comprising the bacteriophage of claim 1.

11. A method for preventing or treating infectious diseases caused by one or more Salmonella strains selected from the group consisting of Salmonella Enteritidis, Salmonella Typhimurium, Salmonella Gallinarum, and Salmonella Pullorum, comprising administering the bacteriophage of claim 1 to animals in need thereof.

12. A method for preventing or treating infectious diseases caused by one or more Salmonella strains selected from the group consisting of Salmonella Enteritidis, Salmonella Typhimurium, Salmonella Gallinarum, and Salmonella Pullorum, comprising administering the bacteriophage composition of claim 6 to animals in need thereof.


SEQUENCE LISTING (20 sequences)

SEQ ID NO: 1 - DNA, 1029 bp, Bacteriophage KCCM11027P
atctcctcaacataggcaacgttaacttcgccggggcgatggctgaccaggctaataccctgaacggagctatcgctagtgcggaggactcatggtcccagttgaagatgactctcgctactagtctggatgtagggtcgctggcggaacctttacgttatattgacgacctgatacaggagataaacgctcaggtcgcatccggtgagttcgtagccgagatgcagatgtggggtgacatggcatccgatgttgggggtgcgatagaggcttcattcgacgcggcgtttgggacggttgcagacgcgttaaacgctttgaactccgcctggacttacaccagtgaaagcattaccggaagcggagaggagaccgcatctacgatagctgagtcggcggcggatgcgctggacttcattgcccaggaatttacagcaatggagcggttttttgaagatatggttaaaggtgctcaggatgcaggacgtcttgtaaaggccgccttgacgccgggagagtcggtagctgaggccaagaaccttaacttccagttagcgcttgctatggatactcaaaggggtgtggctgacctgacacggaaaagtttccgcgaacaagtagaagctcaggaagaccttattgcgctgaagcgagcggcttacgacatcgatgaagaagcagctaaggccgaggggttaggtaagtttaaggtatcaggtacggacagcggttctacaggtgattccgcagacaaggccgctaagaaatccgtcgacgcattcgaacgccagaagaaagccgctgaggatttctattatcagtcaatccaccttaacgacgacgtattccagagatacaagctaaccaagaagagcaacttacaaagctacaggagttctacagcaaccgtctccttagtgaccagcatacgaaaccgcgaagacgcagattatgctcgaggcggatacggccgccaggctgagtagataacgcgagaaagaacgctggaaaaacagttctcgcgatgcg

SEQ ID NO: 2 - DNA, 741 bp, Bacteriophage KCCM11027P
cgaacgccgtcgtaaccgttaatatattttcttttcaattaaggcggcttcggtcgccttttctataagggtcaaataaatgaatctgctttatctcgatactgaaacattttcagaagccgatttaaaaaaagtcggttcctatgcttacgccgaacatccgactaccgaaattgttatctgcacctacgctttcgatgaaggccctgtgcaagtatgggacgccaccgacggcagcgatatgccgcgtgatttgcgtcgggcgatgttgaagctgcaaaaaccagacagcaatctcaaactggtaggccaaaacttccttatgttcgaccgaccagttattaagcattgctgggggttcgaactcctggtagaaaacattatagacactatgatagtagcgttccgacatgccctcccgggttcactggccgcgctgtgtgaggttttaaacattgacgcaagcatggctaaggataaacgtggtaaggcgctgatacagcgattcagtaagcctacgcccaagaactataagattcgacgttatactgccgatacccacccaaaagagtgggcagaatttattgcatacgcaaaaagcgactttacgtccatgcgtgaagtgtataagaaaatgcctaactgggtgaattctgagttcgaaaaccgcgtgcggcacttaaacggggcgattaattaccgaggattaaaggtgttgtggctttgctggaagcggtaaag

SEQ ID NO: 3 - DNA, 997 bp, Bacteriophage KCCM11027P
atctcctcaacataggcaacgttaacttcgccggggcgatggctgaccaggctaataccctgaacggagctatcgctagtgcggaggactcatggtcccagttgaagatgactctcgctactagtctggatgtagggtcgctggcggaacctttacgttatattgacgacctgatacaggagataaacgctcaggtcgcatccggtgagttcgtagccgagatgcagatgtggggtgacatggcatccgatgttgggggtgcgatagaggcttcattcgacgcggcgtttgggacggttgcagacgcgttaaacgctttgaactccgcctggacttacaccagtgaaagcattaccggaagcggagaggagaccgcatctacgatagctgagtcggcggcggatgcgctggacttcattgcccaggaatttacagcaatggagcggttttttgaagatatggttaaaggtgctcaggatgcaggacgtcttgtaaaggccgccttgacgccgggagagtcggtagctgaggccaagaaccttaacttccagttagcgcttgctatggatactcaaaggggtgtggctgacctgacacggaaaagtttccgcgaacaagtagaagctcaggaagaccttattgcgctgaagcgagcggcttacgacatcgatgaagaagcagctaaggccgaggggttaggtaagtttaaggtatcaggtacggacagcggttctacaggtgattccgcagacaaggccgctaagaaatccgtcgacgcattcgaacgccagaagaaagccgctgaggatttctattatcagtcaatccaccttaacgacgacgtattccagaagatacaagctaaccaagagagcaacttacaaagctacaggagttctacagcaaccgtctccttagtgaccagcaatacgaaaccgcgaagacgcagatatgcctcgaggcggatacggccgccaggctgagttagataacgcga

SEQ ID NO: 4 - DNA, 969 bp, Bacteriophage KCCM11027P
aacggtctcattgttgctgaagttgtaatcgcggtcgataagaacactatcaacagcggcatccgccaatcgttgtaacgtagtaaagtcagacagcttaacggagtacttaaatttcttatccgcttccgcacggaaggtgatgtccccaaaagctacccaagcagaatcagaaatgccaccggttgttagaggggtagacgccgcggggataactttcggcagggagccacgccatgcgtaatagttaccgtccccgccatcttctttcggccaaagaaccgctttattggcgtcgttaattcccagggttccgccggttgtgaagttaaaagacgccggagagaaaccggcgtcacgcaagaccgcgggcagcgtcttctgcgtctgcccggttacctggttagtagcgtagtcgatatctgcgccacccgctacaccaccctgtctgccggtgatcacctcggcttcgaaaatctggtgttttttagctacctgtaaatcgtaaagtgacaatacatcaccgcaaccactagacatatgactatcctcttagttaaaaccgttatcgaatccgttagagaacgcacgaccgaaaggcggtacgttgtcgaacttatagaaatccttgtcatagttaaacccggttattttgactgttctgtcatcgccggggtctaccgtagaaacaagaatcatctgagcattatgccttgcttcgttgccgaatgaaaattcagttttcagggcgctattacccgtgtaaatggcttcctgcggtacagaagtcataatcacccgcctgtcgtgcgctccagggactacattaacactttgcacgccgccgtcacgtaacttgagaatcaaagaatggtcatctcccgaggtgaacttaactgggttgtgacagttctacgtgagcccgtttaccgcggttacgtagccgtcgtagtcgcatacgtgaaccct

SEQ ID NO: 5 - DNA, 918 bp, Bacteriophage KCCM11027P
gacgtagagataataggacactattaacggttatgcaagctattttgttattcttatttggcgtctacggtcgcaccggaaaagcgggtggttcccgcctggtgcaactaccaaccagtaaccgactaaccaaagggcattactatgaaacttagcgatttttactacgaagccgaagccgagaaaggcgcgcgcatgccgattcctttaaaagatggtacagattcaggagaatggttgaacgttgtctccccggaggccgatgtcgcggttaaagctatgcgtgccttcaccctggcgtaccgagcggcggtaggtaaattaaaaccgcttcgggataaatgcgaagagcaaaaagacttctcagaatacaatttaaaaatggaagacgcggcaggagacctaaaccgacaattggctctcgaattggtgaatggctggagtctcgatgatgagttcactaaggaaaatcttaagactcttctcacccaatataagcgcctggcggaacatgtagtcgtattccaccacgaacagttgcgtcaattgcaggaaaagtagacgcgttgtttcagtttgcccgttggaacttcataacccgccacgaaaggcgcaagtttgacagtatcgccgatgggcacaaagccgcgcttatcgctatgggggtaataaaagacgcgggagaaacaacgcaggacgccgggcctgaatgcccccctgaactactcaccacttttgagaagtatcgtgatgttaaattcacccgccgcgttgatgacgacggcgtaaagctatacccaagagagcaacttagttggtcagatttagtggcgtatagcactatttcaggtcagatatagggatgtttgaatctgaaattatcatgggcttagacgccatttttgagggtagaaacga

SEQ ID NO: 6 - DNA, 1004 bp, Bacteriophage KCCM11027P
aacttatacgggcaaacacgtgacaacaccccacactacggcaacagaacaacagtacactcgatgcgcaagcagaccccacgtgctgcggaagtaaagcaaccacagttacgtatgattgccaccgaaatggtacacaaataccttggtaacggtgttttcgaagatacgatgacccccaatacccaggccgtgcaatctcttatccgcctggcgcgtgatccggatgtggggggtttaaacctgacggtacgcaacatggataagttacttgccgtgcagaacgaggtcgaagcgtatttcggtgacaaacaggctggagaattttgttacacgtttgatgactataaaaccactatgcaggatatagttagtactatagccgacgctatcttctgtactccatataggcgtggggcggatatccttctcgattttgaacgccctcgcatgggccctgagatggtgttcacccaccgaagcaaggctggtacttccgaaaaatggaccagaacctttaacgatgctcaggtgttcgatagccttaaattctcgtacatagacccaaaaacaaacgttaaagaaacaataacaataccagaaactggtggggttaaaacggagacctacgattcaaaaggtattcgcaactataagcaggctttctgggcagcgcaccgccgccaccagaagaacattttaaagaaaatttcagtgtcgtttaccgccacggaagagggtatctttgctttaccaaatcgtgccattagtgtggttaagggttcacgtatggcgacctacgacggctacgtaaccgcggtaaacgggctcaccgtagaactgtcacaaccagttaagttcacctcgggagatgaccattctttgattctcaagttacgtgacggcggcgtgcaaagtgttaatgtagtccctggagcgcacgacaggcgggtgattatgacttctgtaccgcaggaagccatttacacgggtatagcgccctg

SEQ ID NO: 7 - DNA, 18 bp, Artificial Sequence
  primer
cctgaacggagctatcgc

SEQ ID NO: 8 - DNA, 20 bp, Artificial Sequence
  primer
ggagacggttgctgtagaac

SEQ ID NO: 9 - DNA, 20 bp, Artificial Sequence
  primer
atctgctttatctcgatact

SEQ ID NO: 10 - DNA, 18 bp, Artificial Sequence
  primer
cccgtttaagtgccgcac

SEQ ID NO: 11 - DNA, 18 bp, Artificial Sequence
  primer
tcctcaacataggcaacg

SEQ ID NO: 12 - DNA, 20 bp, Artificial Sequence
  primer
aggagacggttgctgtagaa

SEQ ID NO: 13 - DNA, 19 bp, Artificial Sequence
  primer
aacggtctcattgttgctg

SEQ ID NO: 14 - DNA, 18 bp, Artificial Sequence
  primer
gacggctacgtaaccgcg

SEQ ID NO: 15 - DNA, 19 bp, Artificial Sequence
  primer
gattcctttaaaagatggt

SEQ ID NO: 16 - DNA, 18 bp, Artificial Sequence
  primer
atggcgtctaagcccatg

SEQ ID NO: 17 - DNA, 20 bp, Artificial Sequence
  primer
ccacactacggcaacagaac

SEQ ID NO: 18 - DNA, 19 bp, Artificial Sequence
  primer
cgccgtcacgtaacttgag

SEQ ID NO: 19 - DNA, 19 bp, Artificial Sequence
  primer
cgggcctcttcgctattac

SEQ ID NO: 20 - DNA, 20 bp, Artificial Sequence
  primer
aggcttacccgtcttactgt